(19) United States
(12) Patent Application Publication
(10) Pub. No.: US 2020/0155008 A1
(43) Pub. Date: May 21, 2020

(54) BIOLOGICAL INFORMATION DETECTING APPARATUS AND BIOLOGICAL INFORMATION DETECTING METHOD

(71) Applicant: Hitachi, Ltd., Tokyo (JP)

(72) Inventors: Nobuhiro FUKUDA, Tokyo (JP); Tomoyuki ISHII, Tokyo (JP); Masayoshi ISHIBASHI, Tokyo (JP)

(21) Appl. No.: 16/666,821

(22) Filed: Oct. 29, 2019

(30) Foreign Application Priority Data
Nov. 19, 2018 (JP) 2018-216787

(51) Int. Cl. A61B 5/00; A61B 5/024; A61B 5/16; A61B 5/021

(52) U.S. Cl. CPC A61B 5/0077; A61B 5/02416; A61B 5/486; A61B 5/742; A61B 2576/02; A61B 5/165; A61B 5/7282; A61B 5/02108; A61B 5/7278; A61B 5/4035

(57) ABSTRACT

The biological information detecting apparatus includes a face detecting section detecting the face of a person from an image signal, an expression detecting section detecting an expression of the person from the image signal of the region of the face to calculate an expression feature amount, a pulse wave detecting section detecting a pulse wave of a blood flow of the person from the image signal of the region of the face, a scoring section calculating a score of the expression of the person based on the expression feature amount, a coaching section generating an expression guide, and a display section displaying the expression guide, and the display section further displays biological information indicating a state of the autonomic nerve of the person calculated based on the pulse wave after displaying the expression guide, and the score calculated based on the expression feature amount.

### CLAIM OF PRIORITY

The present application claims priority from Japanese patent application JP 2018-216787 filed on Nov. 19, 2018, the content of which is hereby incorporated by reference into this application.

### BACKGROUND OF THE INVENTION

### 1. Field of the Invention

The present invention relates to an apparatus for detecting biological information.

### 2. Description of the Related Art

As a method of acquiring biological information, there is a technique capable of detecting the information in real time in a non-contact manner by use of a microwave or a camera. In particular, with regard to pulse detection using a camera, miniaturization of camera modules has progressed in recent years, and the modules are mounted on portable terminals including smart phones and are in widespread use. In addition, there are techniques for estimating stress and emotion from biological information as measures to improve work styles and mental health measures in companies.

As a technique for performing pulse detection by image capturing, for example, there is a method of identifying a pulse signal from wavelength fluctuation of a spectrum in JP-2018-086130-A.

In blood pressure measurement in real time, a direct measurement type in which the blood pressure is directly monitored using a catheter is often performed in medicine, but in recent years there is a non-invasive method of performing measurement by pressing a sensor against the artery and converting a change in arterial internal pressure beating against this sensor into an electrical signal. Further, it is known to estimate the blood pressure by the Moens-Korteweg equation, which indicates the relationship between the pulse wave velocity and the incremental elastic modulus of the arterial wall (Tijsseling A. S., Anderson A. (2012) “A. Isebree Moens and D. J. Korteweg: on the speed of propagation of waves in elastic tubes,” BHR Group, Proc. of the 11th Int. Conf. on Pressure Surges (Editor Sandy Anderson), Lisbon, Portugal, October (2012)).

In addition, as a method of estimating emotion using a Russell circle (J. A. Russell, “A circumplex model of affect,” Journal of Personality and Social Psychology, 39 (6), 1161-1178), there is a method of “acquiring the first data corresponding to the physiological data and the second data different from the first data and corresponding to one of the physiological data and the non-physiological data from the subject, calculating a first value indicating the degree of arousal of the subject and a second value indicating the degree of comfort of the subject based on the acquired first data and second data, and thus estimating the subject's emotion from the calculated first value and second value based on the predetermined correspondence between the degree of human arousal and comfort stored in the memory in advance, and the human emotion in JP 2017-144222 A.

### SUMMARY OF THE INVENTION

The above stress estimation and emotion estimation techniques are effective for improving working styles and mental health measures, but monitoring techniques alone cannot promote mental change.

When a person smiles, the function of the parasympathetic nerve is activated and stress is alleviated. Also, it is known that mechanical artificial smiling is effective. Meanwhile, it is difficult to realize the effect only by facial expression training such as smiling in order to promote mental change.

Therefore, a technique is provided to detect a facial expression using a camera and display a guidance so as to guide the user to a smile with a smartphone or a monitor, thereby reducing stress and allowing the user to realize the effect before and after the guidance.

In order to solve the above-mentioned problems, a biological information detecting apparatus that is a representative example of the invention disclosed in the present application includes a face detecting section that detects a face of a person from an image signal of an image captured by a camera, an expression detecting section that detects an expression of the person from the image signal of a region of the face detected by the face detecting section and to calculate an expression feature amount, a pulse wave detecting section that detects a pulse wave of a blood flow of the person from the image signal of the region of the face detected by the face detecting section, a scoring section that calculates a score of the expression of the person based on the expression feature amount, a coaching section that generates an expression guide that induces a change in the expression of the person so as to improve the score, and a display section displaying the expression guide, and the display section further displays biological information indicating a state of an autonomic nerve of the person calculated based on the pulse wave after displaying the expression guide, and the score calculated based on the expression feature amount after displaying the expression guide.

According to one aspect of the present invention, a biological information detecting apparatus capable of more efficiently supporting mental health can be provided by allowing the user to simultaneously grasp the effects of facial expression training and accompanying healing and emotion changes. Problems, configurations, and effects other than those described above will be clarified by the description of the following embodiments.

### BRIEF DESCRIPTION OF THE DRAWINGS

FIG. 1 is a block diagram depicting an example of a configuration of a biological information detecting apparatus according to a first embodiment;

FIG. 2 is a diagram for illustrating an example of a wavelength signal generating section of the biological information detecting apparatus according to the first embodiment;

FIG. 3A is a block diagram for illustrating an example of a wavelength fluctuation detecting section of the biological information detecting apparatus in the first embodiment;

FIG. 3B is a diagram for illustrating an example of a wavelength fluctuation detecting section whose output can be adjusted according to a skin color area in the biological information detecting apparatus in the first embodiment;

FIG. 4 is a diagram for illustrating an example of a pulse wave detecting section of the biological information detecting apparatus according to the first embodiment;

FIG. 5 is a diagram for illustrating an example of a stress index calculating section of the biological information detecting apparatus according to the first embodiment;

FIG. 6 is a diagram for illustrating an example of an expression training section of the biological information detecting apparatus according to the first embodiment;

FIG. 7 is a diagram for illustrating an example of a spatial filter in the first embodiment;

FIG. 8 is a diagram for illustrating an example of a hue-saturation-value (HSV) color space and a designated range of a partial color space in the first embodiment;

FIG. 9 is a diagram for illustrating an example of a method of setting the partial color space in the first embodiment;

FIG. 10 is a diagram for illustrating expression detection and coaching in the first embodiment;

FIG. 11 is a diagram for illustrating an effect on the expression and the living body in the first embodiment.

FIG. 12 is a diagram for illustrating processing timings in the first embodiment;

FIG. 13 is a diagram for illustrating another example of the pulse wave calculating section of the biological information detecting apparatus in the first embodiment;

FIG. 14 is a block diagram depicting an example of a configuration of a biological information detecting apparatus according to a second embodiment;

FIG. 15 is a diagram for illustrating an example of a blood pressure estimating section of the biological information detecting apparatus according to the second embodiment;

FIG. 16 is a diagram for illustrating an example of segment regions and a pulse wave signal obtained from each segment region in the second embodiment;

FIG. 17 is a diagram for illustrating an example of division of a face region and calculation of a pulse wave velocity in the second embodiment;

FIG. 18 is a diagram for illustrating an example of a method of calculating the pulse wave velocity in the second embodiment;

FIG. 19 is a diagram for illustrating another example of the method of calculating the pulse wave velocity in the second embodiment; and

FIG. 20 is a diagram for illustrating an example of psychological circle coordinates of an emotion estimating section in the second embodiment.

### DESCRIPTION OF THE PREFERRED EMBODIMENTS

Hereinafter, embodiments of the present invention will be described based on the figures, but the present invention is not necessarily limited to these embodiments. In the figures for illustrating the embodiments, the same members are denoted by the same reference numerals, and the repeated description thereof will be omitted.

### First Embodiment

In the present embodiment, an example of a biological information detecting apparatus which has a function of detecting a stress index from a face image by using a camera and performs coaching for smiles in parallel thereto, thereby presenting a healing effect will be described.

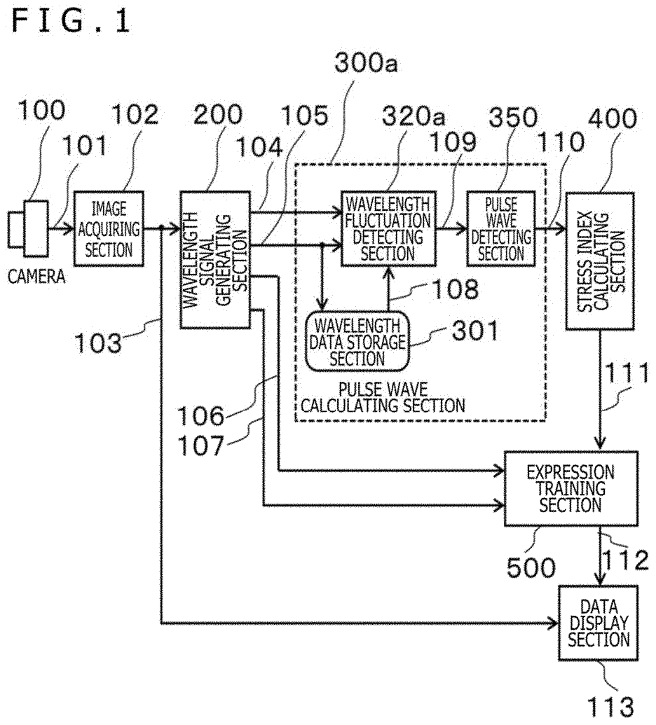

FIG. 1 is a block diagram depicting an example of the configuration of a biological information detecting apparatus according to the first embodiment.

The biological information detecting apparatus according to the present embodiment includes a camera 100, a wavelength signal generating section 200, a pulse wave calculating section 300a, a stress index calculating section 400, an expression training section 500, and a data display section 113.

An image acquiring section 102 receives an imaging data signal 101 acquired from the camera 100 as an input, converts the signal into an RGB signal 103 of an image, and outputs the RGB signal 103. The camera 100 may be, for example, a digital video camera capable of outputting moving images of substantially 30 frames in one second, and the imaging data signal 101 includes a face image of a person to be trained. The wavelength signal generating section 200 receives the RGB signal 103 as an input and outputs a skin color level signal 104, a wavelength data signal or a hue signal 105, a face region signal 106, and a smoothed RGB signal 107.

The pulse wave calculating section 300a receives the skin color level signal 104 and the wavelength data signal or the hue signal 105 as an input and outputs a pulse wave signal 110. The stress index calculating section 400 receives the pulse wave signal 110 as an input and outputs a stress index 111. The expression training section 500 receives the face region signal 106, the smoothed RGB signal 107, and the stress index 111 as an input, and outputs a coaching image 112. The data display section 113 outputs an image obtained by superimposing the RGB signal 103 on the coaching image 112 on a liquid crystal display (LCD).

The pulse wave calculating section 300a includes a wavelength fluctuation detecting section 320a, a pulse wave detecting section 350, and a wavelength data storage section 301. The wavelength fluctuation detecting section 320a receives the skin color level signal 104 and the wavelength data signal or the hue signal 105 as an input and outputs an average wavelength difference data signal 109. The pulse wave detecting section 350 receives the average wavelength difference data signal 109 as an input, detects a pulse wave of a blood flow based thereon, and outputs the pulse wave signal 110. The wavelength data storage section 301 receives the wavelength data signal or the hue signal 105 as an input and outputs a delayed wavelength data signal or a delayed hue signal 108.

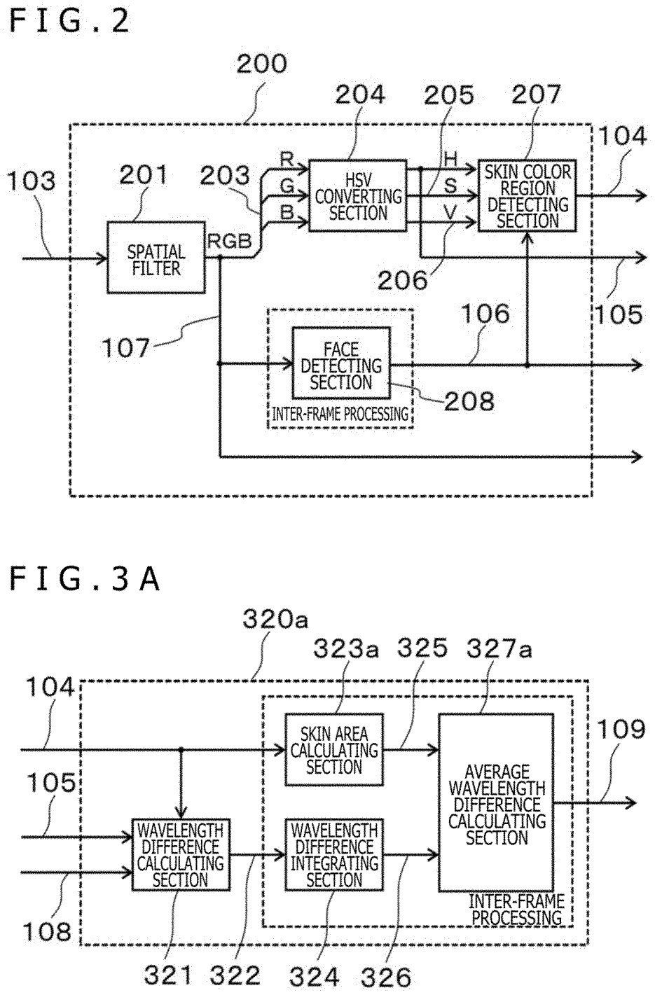

FIG. 2 is a diagram for illustrating an example of the wavelength signal generating section 200 of the biological information detecting apparatus in the first embodiment.

The wavelength signal generating section 200 includes a spatial filter 201, an HSV converting section 204, a skin color region detecting section 207, and a face detecting section 208. The spatial filter 201 receives the RGB signal 103 as an input and outputs the smoothed RGB signal 107 smoothed by, for example, a convolution filter or an average value filter. The HSV converting section 204 receives unpacked signals 203 obtained by decomposing the smoothed RGB signal 107 into red or R, green or G and blue or B signals, and converts the signals into the H signal or hue, that is, the wavelength data signal 105 corresponding to the wavelength, S signal or saturation 205 and V signal or lightness 206.

The skin color region detecting section 207 receives the wavelength data signal 105, S signal or saturation 205 and V signal or lightness 206 as an input, and outputs the skin color level signal 104 indicating a skin color region which is a region on a color space including human skin color. The face detecting section 208 receives the smoothed RGB signal 107 as an input, detects a human face based thereon, and outputs the face region signal 106. The face detection may be performed by a method of dynamically cutting out a face portion from a frame image, for example, as in the known Viola-Jones method or the like, or a method of cutting out by inserting a face in a fixed frame. Here, the face region signal 106 outputs “1” when the face portion is included in the frame, and outputs “0” when the face portion is not included in the frame.

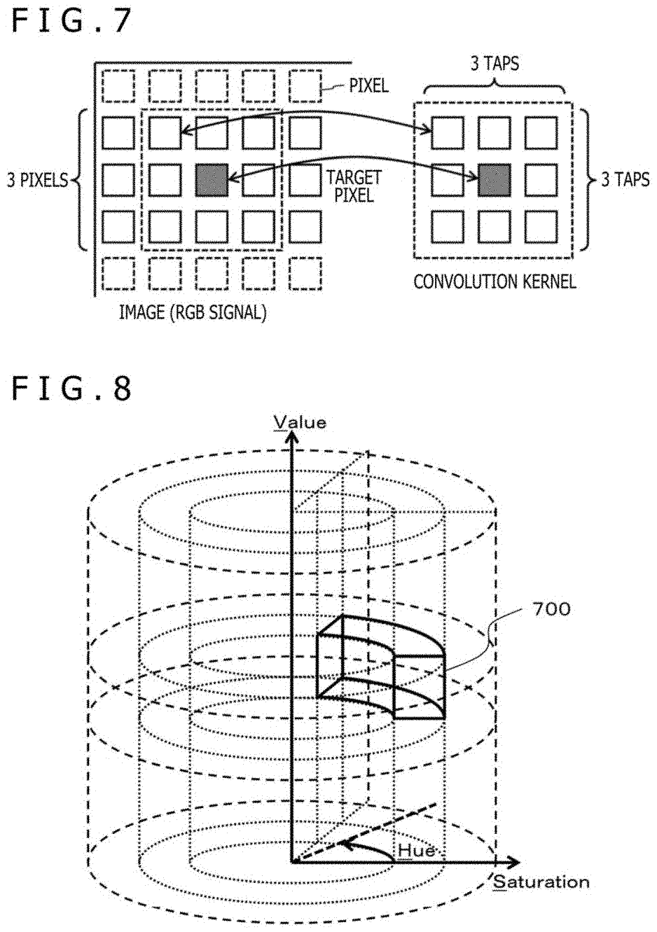

FIG. 7 is a diagram for illustrating an example of the spatial filter in the first embodiment.

FIG. 7 depicts an example in which three taps in length and width, that is, a 3×3 convolution kernel is applied to an image and a value obtained by performing a convolution operation with the kernel around the target pixel of the image becomes the smoothed RGB signal 107. The kernel value is a weighted average coefficient, and their total value has only to be 1.0. Also, a mean value distribution, a Gaussian distribution, or the like can be used for smoothing, for example.

FIG. 8 is a diagram for illustrating an example of the HSV color space and the designated range of the partial color space in the first embodiment.

FIG. 8 represents the HSV color space in cylindrical coordinates. The vertical axis refers to Value, namely lightness, and represents the lightness of the color. The radial axis refers to Saturation, namely chroma which indicates the color depth. The rotation angle refers to Hue, namely color tone. The hue is independent of the intensity and the density and is considered to correspond to the wavelength component of the reflected light, considering that the imaging is done by capturing the reflection of light. Similarly, the lightness can be considered to indicate the intensity of a particular wavelength.

The skin color region detecting section 207 designates a skin color region using a partial color space like a region 700 in FIG. 8 in this HSV color space, and may output “1” as the skin color level signal 104 when the HSV value is included in the skin color region, and may output “0” when the HSV value is not included in the skin color region.

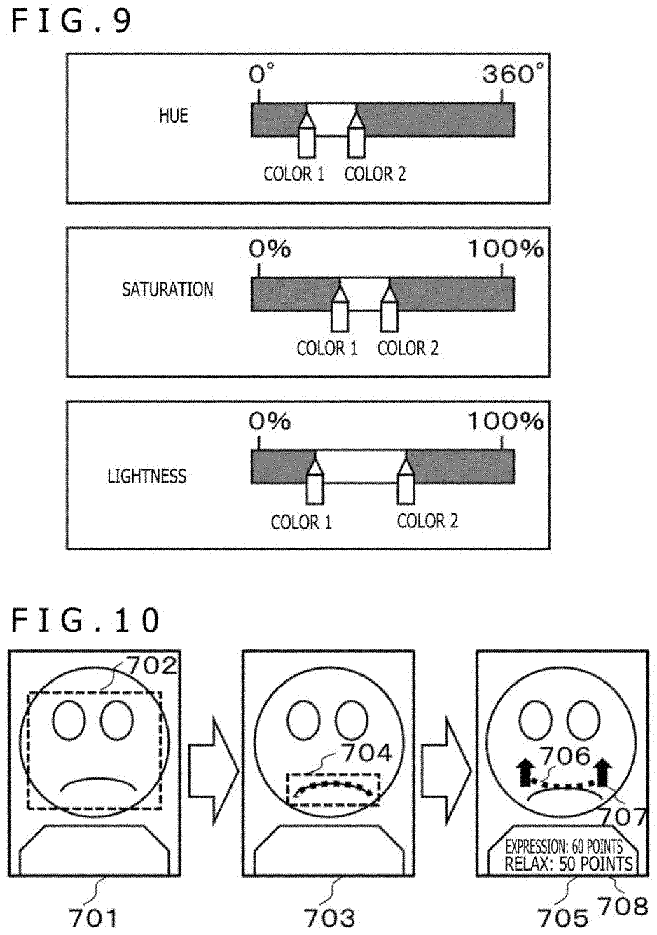

FIG. 9 is a diagram for illustrating an example of a method of setting a partial color space in the first embodiment.

For example, the data display section 113 of the biological information detecting apparatus displays a bar indicating the full range of each of the hue, the saturation, and the lightness, and an icon indicating both ends of the range designated on those bars, for example, “color 1” and “color 2” for designating the hue, as depicted in FIG. 9, and the user may also designate a range by operating those icons using an input device, not depicted, of the biological information detecting apparatus.

For example, for the hue, a bar in the range of 0 degrees to 360 degrees is displayed, and an angle of 0 degrees=360 degrees refers to red, an angle of 120 degrees refers to green, and an angle of 240 degrees refers to blue, and the section designated by the color 1 and color 2, i.e., the range of hue from color 1 to color 2, may be set as the corresponding range as depicted in FIG. 9. Similarly, by defining 0% as light color and 100% as deep color for the saturation, and defining 0% as dark color and 100% as bright color for the lightness, and the range may be specified by designating both ends of the range, for example, saturation 1 and saturation 2 for the saturation, lightness 1 and lightness 2 for the lightness. For example, the color and brightness of the skin to be captured may be largely different depending on a type of illumination used when the camera 100 captures an image of a person and individual differences in the skin color of each person to be captured, but by setting the appropriate range of the hue, the saturation, and the lightness using the setting method described above, the pulse can be appropriately detected in accordance with an image capturing environment, a property of the person to be captured, for example, the color of the skin, and the like.

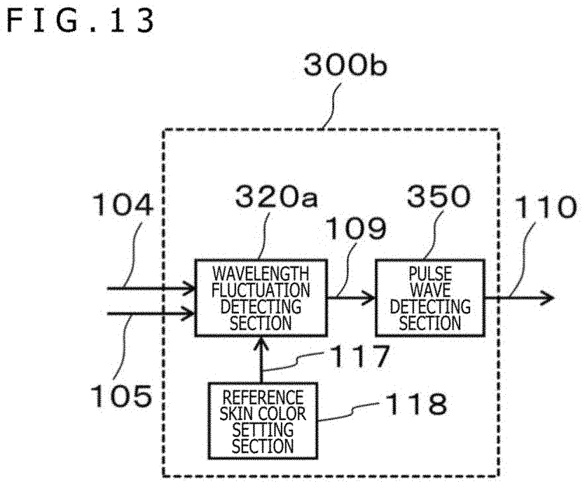

In the configuration of the biological information detecting apparatus in FIG. 1, the pulse wave calculating section 300a may be replaced by a pulse wave calculating section 300b illustrated in FIG. 13.

FIG. 13 is a diagram for illustrating another example of the pulse wave calculating section of the biological information detecting apparatus in the first embodiment.

The pulse wave calculating section 300b depicted in FIG. 13 has the wavelength fluctuation detecting section 320a that outputs the average wavelength difference data signal 109, the pulse wave detecting section 350 that receives the average wavelength difference data signal 109 as an input and outputs the pulse wave signal 110 and a reference skin color setting section 118 that sets the value of a reference skin color wavelength data signal 117 with respect to the wavelength fluctuation detecting section 320a.

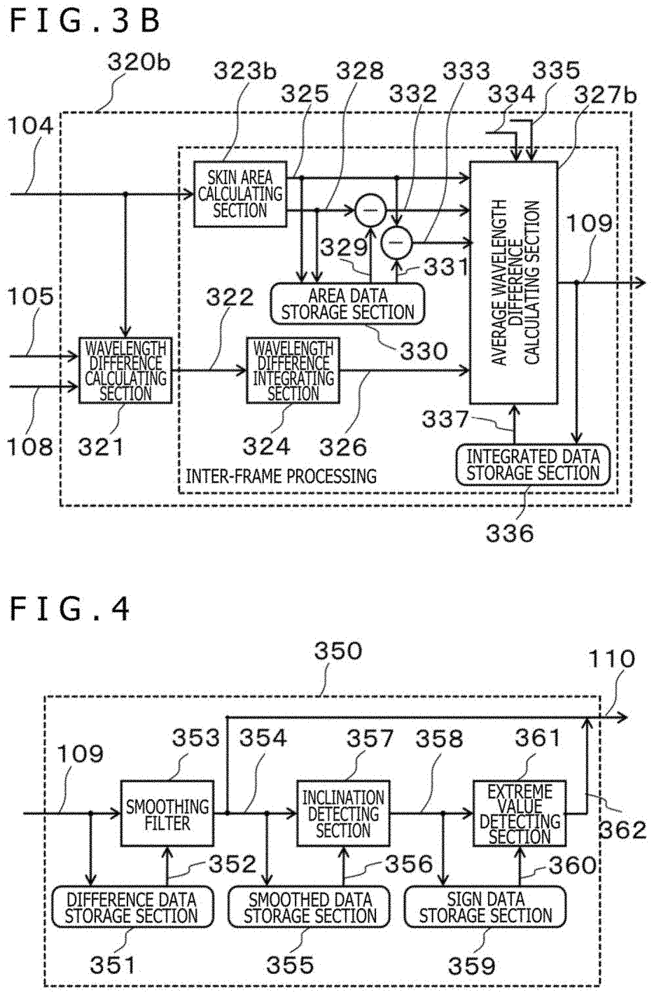

FIG. 3A is a block diagram for illustrating an example of a wavelength fluctuation detecting section of the biological information detecting apparatus according to the first embodiment.

The wavelength fluctuation detecting section 320a includes a wavelength difference calculating section 321, a skin area calculating section 323a, a wavelength difference integrating section 324, and an average wavelength difference calculating section 327a. The wavelength difference calculating section 321 receives the skin color level signal 104 indicating the skin color region, the wavelength data signal 105, and the delayed wavelength data signal 108 as an input, and outputs a wavelength difference data signal 322 calculated from the wavelength data signal 105 and the delayed wavelength data signal 108 that have been input, i.e., the difference signal of the wavelength data signal 105 at each time and the wavelength data signal at a time earlier than each time, namely, the delayed wavelength data signal 108, when the signal of the pixel in the skin color region is input, i.e., “1” is input as the skin color level signal 104, and outputs a value of zero when the signal of the pixel outside the skin color region is input.

The skin area calculating section 323a receives the skin color level signal 104 indicating a skin color region as an input, counts the number of pixels of the skin color region for each frame, and outputs a skin color area signal 325. The wavelength difference integrating section 324 receives the wavelength difference data signal 322 of the skin color region pixel as an input, integrates the wavelength difference for each frame, and outputs an integrated wavelength difference data signal 326. The average wavelength difference calculating section 327a receives the skin color area signal 325 and the integrated wavelength difference data signal 326 as an input, and divides the integrated wavelength difference data by the skin color area, thereby outputting the average wavelength difference data signal 109 between the frames, that is, for all pixels in one frame.

Here, the wavelength fluctuation detecting section 320a in the pulse wave calculating section 300a or 300b may be replaced by a wavelength fluctuation detecting section 320b illustrated in FIG. 3B.

FIG. 3B is a diagram for illustrating an example of a wavelength fluctuation detecting section whose output can be adjusted according to the skin color area in the biological information detecting apparatus in the first embodiment.

The wavelength fluctuation detecting section 320b includes the wavelength difference calculating section 321, a skin area calculating section 323b, an area data storage section 330, the wavelength difference integrating section 324, an integrated data storage section 336, and an average wavelength difference calculating section 327b. The wavelength difference calculating section 321 receives the skin color level signal 104 indicating the skin color region, the wavelength data signal 105, and the delayed wavelength data signal 108, and outputs the wavelength difference data signal 322 calculated from the wavelength data signal 105 and the delayed wavelength data signal 108 that have been input when the signal of the pixel in the skin color region is input, i.e., “1” is input as the skin color level signal 104, and outputs a value of zero when the signal of the pixel outside the skin color region is input.

The skin area calculating section 323b receives the signal 104 including the lightness level indicating the skin color region, counts the number of pixels in the skin color region, that is, pixels of values other than the zero value for each frame, and outputs the skin color area signal 325 indicating the area of the skin color region and a lightness level signal 328 indicating the brightness of the skin color region. The area data storage section 330 receives the skin color area signal 325 and the lightness level signal 328 as an input and outputs a delayed skin color area signal 331 and a delayed lightness level signal 329. The wavelength difference integrating section 324 receives the wavelength difference data signal 322 of the skin color region pixel as an input, integrates the wavelength difference for each frame, and outputs the integrated wavelength difference data signal 326.

The integrated data storage section 336 receives the wavelength difference data signal 109 as an input, holds data for a plurality of frames, and outputs a delayed integrated wavelength data signal 337. The average wavelength difference calculating section 327b receives the skin color area signal 325, an inter-frame lightness level difference signal 332, an inter-frame skin color area difference signal 333, the integrated wavelength difference data signal 326, and the delayed integrated wavelength data signal 337 as an input, and outputs the wavelength difference data signal 109 averaged in the frame by dividing the integrated wavelength difference data by the skin color area.

The inter-frame lightness level difference signal 332 indicates the difference between the lightness level signal 328 of each frame and the lightness level signal 328 of a frame before the current frame, for example, immediately before the current frame, stored in the area data storage section 330, and indicates that a change of the lightness level becomes larger as this difference becomes larger. The inter-frame skin color area difference signal 333 indicates a difference between the skin color area signal 325 of each frame and the skin color area signal 325 of a frame before the current frame, for example, immediately before the current frame, stored in the area data storage section 330, and indicates that a change in the skin color area becomes larger as this difference becomes larger.

The average wavelength difference calculating section 327b may output the delayed integrated wavelength data signal 337, for example, the wavelength difference data signal 109 calculated and output based on the integrated wavelength difference data signal 326 and the skin color area signal 325 of the past frame such as the previous frame, as the wavelength difference data signal 109 for the current frame instead of the average wavelength difference data calculated from the integrated wavelength difference data signal 326 and the skin color area signal 325 of the current frame, when a sudden external light change occurs, that is, when the lightness level difference signal 332 is larger than a lightness level difference threshold 334, or may output an average value of the delayed integrated wavelength data signal 337 and an average wavelength difference data calculated from the integrated wavelength difference data signal 326 and the skin color area signal 325 of the current frame as the wavelength difference data signal 109 regarding the current frame. As a result, the false detection caused by the sudden change of the external light is suppressed.

Similarly, also when a change of the detected skin color region is large, that is, when the skin color area difference signal 333 is larger than a skin color area difference threshold 335, the average wavelength difference calculating section 327b may output the delayed integrated wavelength data signal 337 or the average value of the delayed integrated wavelength data signal 337 and average wavelength difference data calculated from the integrated wavelength difference data signal 326 and the skin color area signal 325 of the current frame as the wavelength difference data signal 109 regarding the current frame, instead of the average wavelength difference data for the current frame.

FIG. 4 is a diagram for illustrating an example of the pulse wave detecting section 350 of the biological information detecting apparatus in the first embodiment.

The pulse wave detecting section 350 includes a difference data storage section 351, a smoothing filter 353, a smoothed data storage section 355, an inclination detecting section 357, a sign data storage section 359, and an extreme value detecting section 361, and performs image processing for each frame. The difference data storage section 351 receives the wavelength difference data signal 109 as an input and outputs a delayed wavelength difference data signal 352. The smoothing filter 353 receives the wavelength difference data signal 109 and the delayed wavelength difference data signal 352 as an input, and outputs a wavelength difference data signal 354 smoothed by wavelength data for a plurality of frames on the continuous time axis. The smoothed data storage section 355 receives the smoothed wavelength difference data signal 354 as an input, holds wavelength difference data for a plurality of frames, and outputs a delayed wavelength difference data signal 356 that has been smoothed.

The inclination detecting section 357 compares the smoothed wavelength difference data signal 354 at a certain time with a signal output from the smoothed data storage section 355, that is, the smoothed wavelength difference data signal 354 at an earlier time, to thereby detect the variation, i.e., inclination, of the smoothed wavelength difference data, outputting a sign data signal 358 for indicating the sign of the inclination. Specifically, the inclination detecting section 357 may compare the smoothed wavelength difference data signals of two consecutive frames, or compare smoothed wavelength difference data signals between the average frames of several consecutive and adjacent frames. In the latter case, for example, the inclination detecting section 357 may compares the average of the wavelength difference data of a plurality of consecutive frames with the average of the wavelength difference data of a plurality of consecutive previous frames before the consecutive frames to calculate the inclination of the difference. The sign data storage section 359 receives the sign data signal 358 as an input, holds sign data of a plurality of frames, and outputs a delayed sign data signal 360.

The extreme value detecting section 361 receives the sign data signal 358 and the delayed sign data signal 360 as an input, and determines an extreme value by regarding the frame in which the sign of inclination changes from a positive value to a negative value, that is, a change in the difference according to the time turns from increase to decrease, as a frame of the maximum value, and by regarding the frame in which the sign changes from a negative value to a positive value, that is, the change in the difference according to the time turns from decrease to increase, as a frame of the minimum value, and outputs, for example, the maximum value or minimum value as a pulse wave extreme value signal 362. The pulse wave detecting section 350 places the pulse wave extreme value signal 362 on the smoothed wavelength difference data signal 354 and outputs the resultant signal as the pulse wave signal 110. Alternatively, the extreme value detecting section 361 may output information indicating the timing at which the maximum value or the minimum value is detected.

As described above, the smoothing filter 353 makes the difference data signal smooth, thereby preventing erroneous detection of a pulse due to a minute change of the difference data signal caused by noise or the like. The inclination detecting section 357 detects a change or inclination of difference data between frames adjacent to each other, and the extreme value detecting section 361 detects the maximum value or minimum value of the difference data based on the result, thereby accurately generating a pulse signal. When the inclination detecting section 357 obtains the difference between the average frames of a plurality of consecutive and adjacent frames, erroneous detection of a pulse is prevented as in the above-described smoothing.

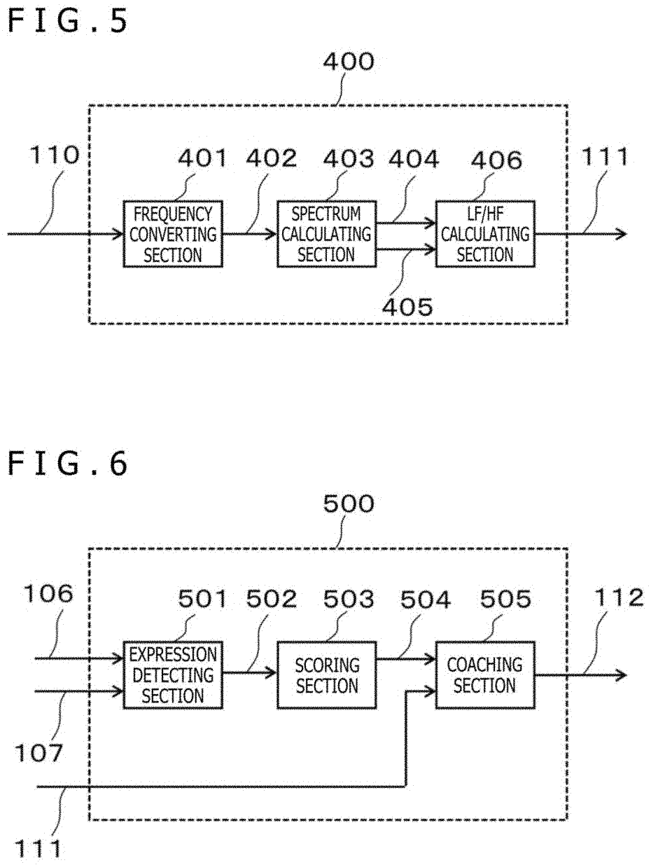

FIG. 5 is a diagram for illustrating an example of the stress index calculating section 400 of the biological information detecting apparatus in the first embodiment.

The stress index calculating section 400 includes a frequency converting section 401, a spectrum calculating section 403, and an LF/HF calculating section 406. The frequency converting section 401 receives the pulse wave signal 110 as an input, converts the frequency by regarding the period of time between the minimum values as the R wave interval or RRI of the heartbeat, for example, and outputs a frequency signal 402. The spectrum calculating section 403 receives the frequency signal 402 as an input and outputs a high frequency signal or HF 404 and a low frequency signal or LF 405. The LF/HF calculating section 406 receives the high frequency signal or HF 404 and the low frequency signal or LF 405 as an input, and outputs the stress index 111.

Here, the LF/HF is called a stress index and can be also used to detect a stress state. For example, the LF is the total value or integrated value of signal intensities in the 0.05-Hz to 0.15-Hz band, and the HF is the total value or integrated value of signal intensities in the 0.15-Hz to 0.40-Hz band.

In other words, the stress index is the ratio LF/HF of the magnitude of the component LF of a relatively low frequency band, e.g., 0.05 Hz to 0.15 Hz, to the magnitude of the component HF of the band, e.g., 0.15 Hz to 0.40 Hz, having frequencies higher than the LF in fluctuation of the pulse or heartbeat, interval calculated from the pulse wave. This is an example of biological information indicating the state of a person's autonomic nerve, and another example of such biological information is blood pressure as well as the LF, HF, and LF/HF described above. The blood pressure will be described in the second embodiment. By using these pieces of information, the effects of the coaching described later can be quantified and evaluated.

FIG. 6 is a diagram illustrating an example of an expression training section of the biological information detecting apparatus according to the first embodiment.

The expression training section 500 includes an expression detecting section 501, a scoring section 503, and a coaching section 505. The expression detecting section 501 receives the face region signal 106 and the smoothed RGB signal 107 as an input, and detects a human facial expression based on these signals, thereby outputting an expression signal 502 indicating a feature of the facial expression. The scoring section 503 receives the expression signal 502 as an input, and calculates and outputs a score 504 of the expression based thereon. The coaching section 505 receives the score 504 and the stress index 111 as an input, and outputs the coaching image 112 for improving the score based thereon.

For example, the expression detecting section 501 may detect the expression of a user based on a ratio calculated from the position of a feature point in a captured image of the user's face, for example, a point where a feature such as a facial expression of the user appears, such as tails of eyes, a nose or an angle of a mouth. In that case, the scoring section 503 may calculate the score so as to make the score higher as the calculated ratio becomes closer to a predetermined ratio, for example, so-called golden ratio or platinum ratio of smile. In that case, the coaching section 505 may generate a facial expression guide that induces a change in facial expression so as to improve the score, and the data display section 113 may display the facial expression guide. Thereby, an appropriate target can be presented, and the user can be guided there.

FIG. 10 is a diagram illustrating facial expression detection and coaching in the first embodiment.

An image 701 is an image obtained by capturing an image of a face, and for example, a portion in which the face is detected in the image is a rectangle 702. An image 703 depicts a state in which the mouth is detected as a feature point from the detected face. At that time, when the smile having the mouth created based on the golden ratio or platinum ratio of the smiling face from the detected face is regarded as an ideal smile, the scoring section 503 calculates the deviation of the detected face from the ideal smile, which is indicated by 704. Then, the coaching section 505 generates an image 705 made by superimposing the deviation amount 706 of the detected face from the ideal smiling face, an image or facial expression guide, guiding to an ideal mouth 707, and an index 708 that scores the expression and the degree of relaxation, for example, on an image of the face of the user whose image is currently being captured, that is, in real time, by the camera 100, for example, as the coaching image 112. Such a coaching image 112 is displayed by the data display section 113. Thus, the user can easily grasp how to change the facial expression in order to improve the score.

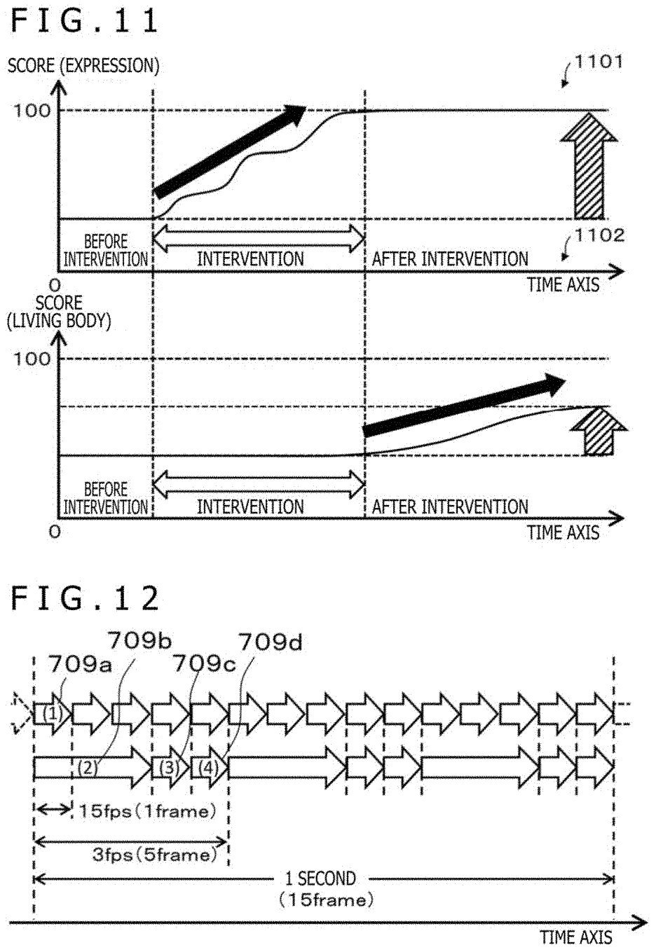

FIG. 11 is a diagram for illustrating the effects on the facial expression and the living body in the first embodiment.

The example of FIG. 11 indicates a graph 1101 displaying temporal transition of the score of the expression obtained by calculating the degree of coincidence between the detected mouth and the ideal mouth as a score of the expression, and a graph 1102 displaying temporal transition of the biological information obtained by scoring a relaxation state using a stress index, for example. These are visual representations of the effects before and after the coaching. Also, these graphs may be displayed as the index 708.

In the example of FIG. 11, intervention means display of the coaching image 112 by the coaching section 505. After the intervention is started and while the intervention is being performed, i.e., while the coaching image 112 is displayed, the user refers to the coaching image 112 and changes one's own expression. For example, a shape of the mouth is changed so as to approach the mouth guide image created on the basis of the golden ratio or the platinum ratio. This allows the facial expression score to rise while the intervention is taking place. Meanwhile, the score of biological information changes, for example, in a relaxed state, under the influence of the change in the expression. The change in the score of this biological information appears later than the change in the score of the expression.

Although an example is depicted which displays the change of the score of the relaxation state using the stress index as biological information which indicates the state of a user's autonomic nerve in FIG. 11, change of other biological information may be displayed. For example, a score indicating the user's emotion estimated based on the LF, HF, LF/HF or blood pressure, or at least one of these may be displayed. The estimation of blood pressure and emotion will be described in the second embodiment.

Such display allows the user to visually grasp the effects of coaching.

FIG. 12 is a diagram for illustrating processing timing in the first embodiment.

Process (1) denoted by 709a in the upper part of the figure, process (2) denoted by 709b, process (3) denoted by 709c and process (4) denoted by 709d in the lower part depict a series of processing from pulse wave detection to stress index calculation and three processes of the expression training section, namely, process (2) in the expression detecting section, the process (3) in the scoring section, and process (4) in the coaching section. Process (1) and process (2) to process (4) may be preferably performed in the same frame, but depending on the processing capability of the processing device for implementation, the processing may span different frames. Therefore, for example, when process (1) is capable of processing 15 frames per second and process (2) has a processing load of three frames and process (3) and process (4) each have a processing load within one frame, the processes may be synchronized as depicted in FIG. 12. That is, in the example of FIG. 12, process (1) of the first three frames is synchronized with process (2), and process (1) of the next one frame is synchronized with process (3), process (1) of further next one frame is synchronized with process (4), and the respective processes are synchronized such that the same processing is repeated.

According to the first embodiment described above, a biological information detecting apparatus can be provided which can more efficiently support mental health by allowing the user to simultaneously grasp the effect of facial expression training and the accompanying change in stress.

### Second Embodiment

In the first embodiment, an example of a biological information detecting apparatus which has a function of detecting a stress index from a face image using a camera, performs smile coaching in parallel thereto, and presents a healing effect has been described, and now, a biological information detecting apparatus that detects emotion and provides a healing effect will be described in the second embodiment. Except for differences described below, the sections of the biological information detecting apparatus of the second embodiment have the same functions as the sections denoted by the same reference numerals of the first embodiment depicted in FIGS. 1 to 13, and therefore their description will be omitted.

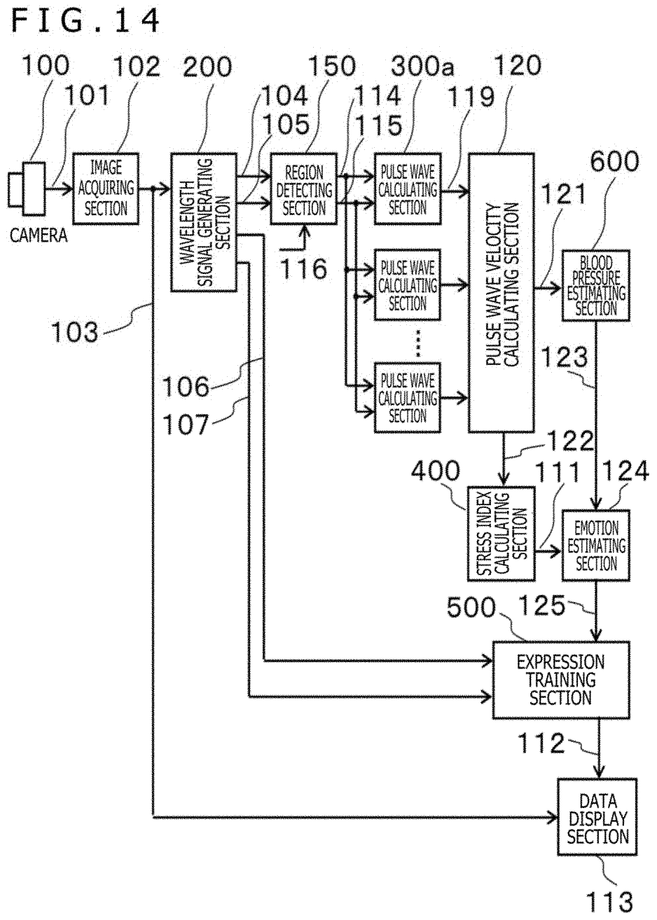

FIG. 14 is a block diagram depicting a configuration example of the biological information detecting apparatus in the second embodiment.

The biological information detecting apparatus according to the second embodiment includes the camera 100, the wavelength signal generating section 200, a region detecting section 150, a plurality of pulse wave calculating sections 300a, a pulse wave velocity calculating section 120, a blood pressure estimating section 600, the stress index calculating section 400, an emotion estimating section 124, the expression training section 500, and the data display section 113.

Here, the image acquiring section 102, the wavelength signal generating section 200, the expression training section 500, and the data display section 113 have the same configurations as in the first embodiment. In addition, each of the plurality of pulse wave calculating sections 300a has the same configuration as the pulse wave calculating section 300a of the first embodiment.

The region detecting section 150 receives the skin color level signal 104 and the wavelength data signal or the hue signal 105 as an input, subdivides the camera screen into a plurality of segment regions by a division number parameter 116, and passes a segment skin color level signal 114 and a segment wavelength data signal 115 to the pulse wave calculating section 300a corresponding to each segment region. The pulse wave velocity calculating section 120 calculates a pulse wave velocity on the basis of a segment pulse wave signal 119 output from the pulse wave calculating section 300a corresponding to each segment region, and outputs a pulse wave velocity signal 121 and an average pulse wave signal 122. The emotion estimating section 124 uses an estimated blood pressure value 123 and the stress index 111 as input signals, and outputs an emotion signal 125.

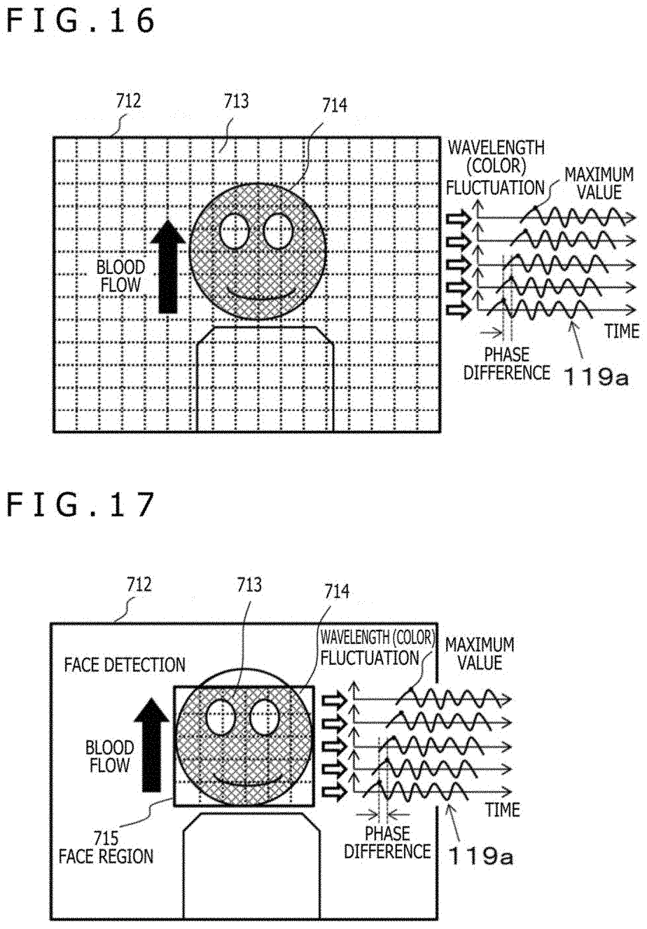

FIG. 16 is a diagram for illustrating an example of the segment region and the pulse wave signal obtained from each segment region in the second embodiment.

To be specific, FIG. 16 depicts an example of an average segment pulse wave signal 119a obtained by dividing a frame image into a plurality of segment regions and by collecting signals from a plurality of segment regions having the same position in the vertical direction, and is a figure illustrating the basic way of thinking for calculating the pulse wave velocity. In FIG. 16, a frame image 712 is represented by a thick solid rectangle.

Here, a segment region 713 refers to each portion when the frame image 712 is divided into a plurality of portions. In the example of FIG. 16, the frame image 712 is divided into a plurality of rectangular regions as indicated by broken lines, and each region becomes one segment region 713. In this example, a plurality of segment regions having the same position in the vertical direction means a plurality of segment regions 713 having the same coordinate value in the vertical direction when each segment region is identified by a coordinate value in the horizontal direction or the right-left direction and a coordinate value in the vertical direction or the up-down direction, for example, in other words, means a single line of the segment regions 713 arranged in the horizontal direction.

In the frame image 712, an image of a person is displayed, and it is depicted that a skin color region 714 or a hatched display portion is present in the face portion of the person.

In FIG. 16, the skin color region 714 refers to a region composed of pixels for which the segment skin color level signal 114 is “1.” The segment pulse wave signal 119 is generated by using the wavelength difference data signal 109 calculated based on the area or the number of pixels of the skin color region 714 included in the segment region 713 and the segment pulse wave signal 119 of the pixels of the skin color region 714. Accordingly, the segment pulse wave signal 119 cannot be obtained from the segment region 713 which does not include the skin color region 714. In addition, also when the area of the skin color region 714 included in one segment region 713 is small, the accurate segment pulse wave signal 119 cannot be obtained. Therefore, the segment pulse wave signal 119 cannot be generated for the segment region 713 where the area ratio of the skin color region 714 to the segment region 713 is equal to or less than a predetermined value, for example, 50% or less, which means generation failed.

Furthermore, as depicted in FIG. 16, the blood in the human face flows from the side closer to the heart to the far side, that is, from the lower side to the upper side or in a direction of a thick arrow. Hence, the segment pulse wave signals 119 having waveforms in which the phases are substantially the same are obtained from the plurality of segment regions 713 aligned in the horizontal direction at the same position in the vertical direction among the segment regions 713 including the skin color region 714, for example, the segment region 713 hatched in FIG. 16. Meanwhile, among the segment regions 713 including the skin color region 714, phase differences occurs in the waveforms of a plurality of segment wavelength data signals 115 obtained from the segment regions 713 having different positions in the vertical direction. This phase difference is nothing but the phase difference of the pulse wave when blood flow propagates in the blood vessel as the heart beats, namely that of the segment pulse wave signal 119.

In addition, the average segment pulse wave signal 119a obtained by averaging the segment pulse wave signals 119 collected from the respective segment regions 713 corresponding to each vertical position is depicted on the outer right side of the frame image 712 in FIG. 16. Further, a time or a time specified by a frame number or the like at which the average segment pulse wave signal 119a becomes an extreme value is output as an average pulse wave extreme value signal 362a.

At this time, the pulse wave velocity (V) can be calculated by using a phase difference time period Δt of the two average segment pulse wave signals 119a having different positions of the segment region 713 in the vertical direction and the vertical distance ΔL. That is, the pulse wave velocity (V) is calculated by the equation V=ΔL/Δt. In addition, the phase difference time period Δt of these two average segment pulse wave signals 119a may be obtained simply as, for example, the time difference between the respective average pulse wave extreme value signals 362a corresponding to the two average segment pulse wave signals 119a.

The average segment pulse wave signal 119a is preferably an average of all of the segment pulse wave signals 119 obtained from the segment regions 713 corresponding to each position in the vertical direction, but the average segment pulse wave signal 119a may be the segment pulse wave signal 119 obtained from one segment region 713 corresponding to each position in the vertical direction. However, in general, the accuracy can be improved by using an averaged measurement value.

FIG. 17 is a diagram for illustrating an example of division of a face region and pulse wave velocity calculation in the second embodiment.

To be specific, FIG. 17 is a diagram illustrating a basic concept of pulse wave velocity calculation, as well as an example of the average segment pulse wave signal 119a that is the average of the segment pulse wave signals 119 obtained by dividing a face region 715 into the plurality of segment regions 713 and collecting signals from the plurality of segment regions 713 having the same position in the vertical direction. FIG. 17 depicts that the face region 715 detected by the face detecting section 208 is displayed as a thick solid rectangle and the face region 715 is divided into the plurality of segment regions 713 by broken lines. Furthermore, it is depicted that the skin color region 714 or a hatched display part is present in the face region 715.

FIG. 17 differs from FIG. 16 in that the segment regions 713 in which the segment pulse wave signals 119 are sought are not set to the entire frame image 712 and are limited to the part of the face region 715 detected by the face detecting section 208. Except for this point, the description of FIG. 17 is the same as the description of FIG. 16, and thus, the description will be omitted.

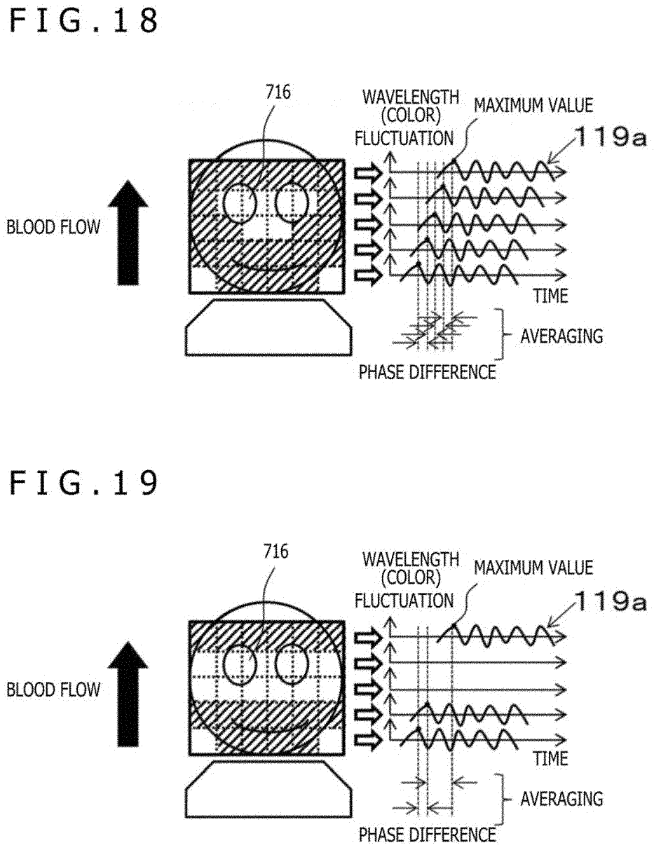

FIG. 18 is a diagram for illustrating an example of a method of calculating the pulse wave velocity in the second embodiment.

To be specific, FIG. 18 is a diagram illustrating the calculation method of the pulse wave velocity in the case where a pulse wave signal missing segment 716 is included in a part of the plurality of segment regions 713 arranged in the horizontal direction and having the same positions in the vertical direction. Here, the pulse wave signal missing segments 716 refer to the segment regions 713 in which the segment pulse wave signal 119 cannot be obtained, and are depicted as white segment regions 713 in FIG. 18. Further, the hatched segment regions 713 in FIG. 18 represent segment regions 713 where the segment pulse wave signal 119 can be obtained.

As described above, in order to calculate the pulse wave velocity, first, an average value of the segment pulse wave signals 119 obtained from the plurality of segment regions 713 having the same positions in the vertical direction and different positions in the horizontal direction, that is, the average segment pulse wave signal 119a is calculated. FIG. 18 depicts an example in which the pulse wave signal missing segments 716 are present in a part of the segment regions 713 in the horizontal direction, but the segment regions 713 where the segment pulse wave signal 119 can be acquired are also present there. In such a case, the average segment pulse wave signal 119a can be obtained by averaging the segment pulse wave signals 119 of the segment regions 713 where the segment pulse wave signals 119 can be obtained.

To be specific, in the example of FIG. 18, with regard to the segment regions 713 arranged horizontally in the second position from the top in the vertical direction, although four out of the six segment regions 713 are the pulse wave signal missing segments 716, the segment pulse wave signals 119 are obtained from the remaining two segment regions 713. In such a case, the pulse wave velocity calculating section 120 can obtain the average segment pulse wave signal 119a for the position in the vertical direction by averaging the segment pulse wave signals 119 from these two segment regions 713.

As described above, when the average segment pulse wave signal 119a at each position in the vertical direction is obtained, the average pulse wave extreme value signal can be obtained from each of the signals. Then, an average value Ave(Δt) can be obtained by averaging the phase difference time periods Δt of the average pulse wave extreme value signals between the positions adjacent to each other in the vertical direction. At this time, the pulse wave velocity (V) can be obtained by the equation V=ΔL/Ave(Δt).

FIG. 19 is a diagram for illustrating another example of the method of calculating the pulse wave velocity in the second embodiment.

To be specific, FIG. 19 is a diagram for illustrating a method of calculating the pulse wave velocity in the case where a position where all the horizontally arranged segment regions 713 are the pulse wave signal missing segments 716 is present in the vertical direction. In the example of FIG. 19, in the second and third positions in the vertical direction from the top, all the horizontally arranged segment regions 713 are the pulse wave signal missing segments 716, and therefore, the average segment pulse wave signals 119a cannot be obtained regarding these positions in the vertical direction. However, for the first, fourth and fifth positions in the vertical direction from the top, the average segment pulse wave signals 119a are acquired.

In such a case, the pulse wave velocity calculating section 120 obtains the phase difference time period Δt1 per one unit of vertical distance from the average segment pulse wave signals 119a at the first and fourth positions in the vertical direction from the top, and further, a phase difference time period Δt2 is obtained from the average segment pulse wave signals 119a at the fourth and fifth positions in the vertical direction from the top. Then, if an average value of these phase difference time periods Δt1 and Δt2 is expressed as Ave(Δt1, Δt2), the pulse wave velocity (V) can be obtained by the equation V=ΔL/Ave(Δt1, Δt2).

As described above, even in the case where there are positions in the vertical direction where all the horizontally aligned segment regions 713 are the pulse wave signal missing segments 716, if the average segment pulse wave signals 119a are acquired at positions on the upper/lower side thereof in the vertical direction, the phase difference time period Δt per one unit of vertical distance can be determined by using the signals. Thus, the pulse wave velocity (V) can be obtained.

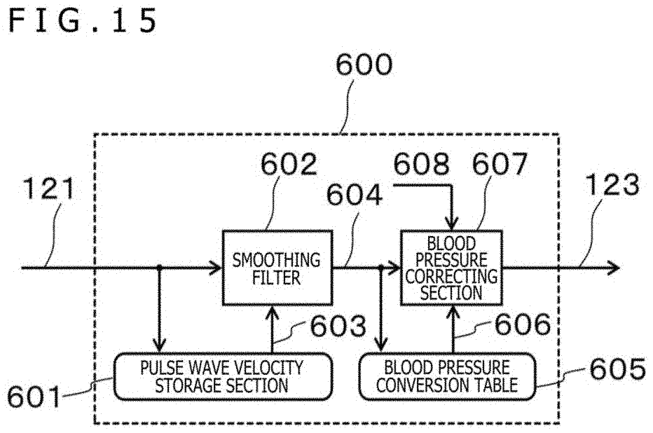

FIG. 15 is a diagram for illustrating an example of a blood pressure estimating section of the biological information detecting apparatus according to the second embodiment.

As depicted in FIG. 15, the blood pressure estimating section 600 includes a pulse wave velocity storage section 601, a smoothing filter 602, a blood pressure conversion table 605, and a blood pressure correcting section 607.

Here, the pulse wave velocity storage section 601 stores the value of the pulse wave velocity signal 121 input for a plurality of frames, and outputs a delayed pulse wave velocity signal 603. In addition, the smoothing filter 602 averages the pulse wave velocity signals 121 and the delayed pulse wave velocity signals 603 that have been input for a plurality of frames, and outputs a smoothed pulse wave velocity signal 604.

When the smoothed pulse wave velocity signal 604 is input, the blood pressure conversion table 605 searches its own table and outputs a blood pressure conversion signal 606 that is a source of blood pressure. According to the Moens-Korteweg equation etc., the diastolic blood pressure value (P) is proportional to the square of the pulse wave velocity (PWV). That is, P=c·PWV² is satisfied. However, this proportionality constant c depends on various types of biological information or age, gender, blood vessel radius, blood density, etc. of the subjects. Therefore, the blood pressure conversion table 605 receives the value of the smoothed pulse wave velocity signal 604 as the pulse wave velocity (PWV), and outputs the blood pressure value for representative biological information determined in advance as the blood pressure conversion signal 606.

The blood pressure correcting section 607 receives the smoothed pulse wave velocity signal 604, the blood pressure conversion signal 606, and a blood pressure correction parameter 608 as an input, and corrects the blood pressure conversion signal 606, thus outputting the estimated blood pressure value 123. Here, the blood pressure correction parameter 608 is a numerical value necessary to determine the proportionality constant c, and is, for example, of age, gender, a blood vessel radius, blood density, and the like. That is, the blood pressure correcting section 607 corrects the blood pressure value obtained by the blood pressure conversion table 605 for the representative biological information according to the biological information of the subject.

In the present embodiment, although the blood pressure estimating section 600 estimates the blood pressure value of the subject by using the pulse wave velocity signal 121, the blood pressure conversion table 605, and the blood pressure correction parameter 608, the estimated blood pressure value 123 of the subject may be calculated by a mathematical expression model using the Moens-Korteweg equation etc.

As described above, according to the second embodiment, the plurality of segment wavelength data signals 115 obtained from the plurality of segment regions 713 including the skin color region 714 is generated based on the segment wavelength data signal 115 corresponding to the hue (H) obtained from the pixels included in the skin color region 714. In this case, the influence of lightness (V) and saturation (S) on the segment wavelength data signal 115 is eliminated. That is, in the second embodiment, the estimated blood pressure value 123 is calculated using the plurality of segment wavelength data signals 115 from which the influence of the lightness (V) and the saturation (S) is eliminated. Accordingly, in the second embodiment, the estimated blood pressure value 123 is obtained in which the influence of the external light, namely the influence of the lightness (V) and the saturation (S) is eliminated.

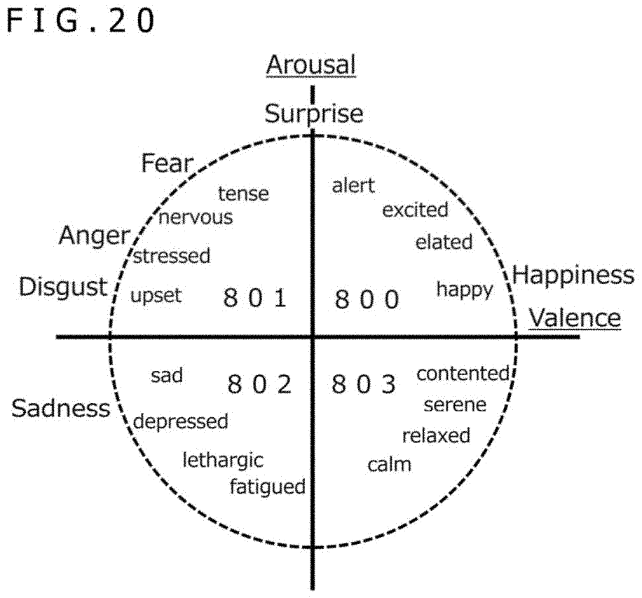

FIG. 20 is a diagram for illustrating an example of the psychological circle coordinates of the emotion estimating section in the second embodiment.

The vertical axis in the figure is the awakening axis or Arousal in the emotion estimating section 124, and the horizontal axis is the comfort/discomfort axis or Valence, and the first quadrant 800 is set to “joy,” the second quadrant 801 is set to “anger,” the third quadrant 802 is set to “sadness,” and the fourth quadrant 803 is set to “comfort” to express emotions. Here, the association of each axis with the biological information may be performed by obtaining the correlation with the psychological circle, here, Russell circle, from experimental results, or may be defined from the psychological factor. For example, the awakening axis may be a function or the like with at least one of the stress index (LF/HF), HF, and LF as a parameter, and the comfort/discomfort axis may be a blood pressure axis, the LF, or the like.

Also, the obtained emotion may be monitored to enter, for example, the quadrant of relaxation, and in FIG. 20, the emotion may be expressed as a distance to a relaxation region in the fourth quadrant 803, for example.

To be specific, for example, with the LF applied to the horizontal axis or the comfort/discomfort axis and the HF applied to the vertical axis or the awakening axis in FIG. 20, the emotion corresponding to the position where the calculated values of LF and HF are plotted may be estimated as the user's emotion at the time point. When the relaxation state is the target, the distance or the reciprocal thereof from the plotted position to the position corresponding to the relaxation state may be displayed in the graph 1102 of FIG. 11 as a score of the relaxation state. Alternatively, blood pressure may be applied to the horizontal axis or the comfort/discomfort axis and the LF/HF may be applied to the vertical axis or the awakening axis in FIG. 20 to perform the same processing as described above.

According to the above configuration, a biological information detecting apparatus capable of more efficiently supporting mental health by allowing the user to simultaneously grasp the effect of facial expression training and the accompanying change in emotion can be provided.

The present invention is not limited to the embodiments described above, and includes various modifications. For example, the embodiments described above have been described in detail for better understanding of the present invention, and are not necessarily limited to those having all the configurations of the description. Further, part of the configuration of one embodiment can be replaced with a configuration of another embodiment, and a configuration of another embodiment can be added to the configuration of one embodiment. In addition, with respect to part of the configuration of each embodiment, addition of other configurations, deletion, and replacement can be carried out.

Further, each of the configurations, functions, processing sections, processing devices, etc. described above may be accomplished by hardware, for example, by designing part or all of them with an integrated circuit. Further, each configuration, function, and the like described above may be achieved by software by the processor interpreting and executing a program that fulfills each function. Information such as programs, tables, and files for fulfilling each function can be stored in a storage device such as a nonvolatile semiconductor memory, hard disk drive, and solid state drive (SSD), or computer readable non-transitory data storage medium such as an integrated circuit (IC) card, a secure digital (SD) card, or a digital versatile disk (DVD).

Further, the control lines and the information lines that are considered to be necessary for description are depicted, and not all the control lines and the information lines in the product are necessarily depicted. In practice, almost all configurations may be considered to be mutually connected.

What is claimed is:

1. A biological information detecting apparatus comprising:
a face detecting section configured to detect a face of a person from an image signal of an image captured by a camera;
an expression detecting section configured to detect an expression of the person from the image signal of a region of the face detected by the face detecting section and to calculate an expression feature amount;
a pulse wave detecting section configured to detect a pulse wave of a blood flow of the person from the image signal of the region of the face detected by the face detecting section;
a scoring section configured to calculate a score of the expression of the person based on the expression feature amount;
a coaching section configured to generate an expression guide that induces a change in the expression of the person so as to improve the score; and
a display section configured to display the expression guide,
wherein the display section further displays biological information indicating a state of an autonomic nerve of the person calculated based on the pulse wave after displaying the expression guide, and the score calculated based on the expression feature amount after displaying the expression guide.

2. The biological information detecting apparatus according to claim 1, further comprising:
a stress index calculating section configured to calculate a stress index indicating a degree of stress of the person as the biological information based on the pulse wave.

3. The biological information detecting apparatus according to claim 2,
wherein the stress index is a ratio of magnitude of a component of a first frequency band to magnitude of a component of a second frequency band higher than the first frequency band in fluctuation of a pulse interval calculated from the pulse wave.

4. The biological information detecting apparatus according to claim 2, further comprising:
a blood pressure estimating section configured to estimate blood pressure of the person as the biological information based on the pulse wave; and
an emotion estimating section configured to estimate emotion of the person based on at least one of magnitude of a component of a first frequency band, magnitude of a component of a second frequency band higher than the first frequency band in fluctuation of a pulse interval calculated from the pulse wave, a ratio of the magnitude of the component of the first frequency band to the magnitude of the component of the second frequency band, and the blood pressure,
wherein the display section displays the estimated emotion.

5. The biological information detecting apparatus according to claim 4,
wherein the blood pressure estimating section estimates the blood pressure of the person based on a phase difference between a pulse wave detected from the image signal of a first portion of the region of the face and a pulse wave detected from the image signal of a second portion of the region of the face that is located above the first portion, and a distance between the first portion and the second portion.

6. The biological information detecting apparatus according to claim 1,
wherein the pulse wave detecting section converts the image signal of the region of the face into a value in an HSV color space, and detects the pulse wave based on fluctuation of a wavelength in a region including a skin color of the person in the HSV color space.

7. The biological information detecting apparatus according to claim 1,
wherein the scoring section calculates the score so as to make the score higher as a ratio calculated based on a position of a feature point of the face of the person becomes closer to a predetermined ratio.

8. The biological information detecting apparatus according to claim 7,
wherein the predetermined ratio is a golden ratio or a platinum ratio of a smile.

9. The biological information detecting apparatus according to claim 7,
wherein the coaching section generates information for guiding the position of the feature point of the face of the person whose image has been captured by the camera to a position where the score is increased, as the expression guide, and
the display section superimposes the expression guide on an image of a current face of the person, the image being captured by the camera, to display the expression guide.

10. The biological information detecting apparatus according to claim 1,
wherein the display section displays at least a change in the score and the biological information in a time zone after displaying the expression guide.

11. A method for detecting biological information performed by a biological information detecting apparatus, the method comprising:
a face detecting step of detecting a face of a person from an image signal of an image captured by a camera;
an expression detecting step of detecting an expression of the person from the image signal of a region of the face detected in the face detecting step to calculate an expression feature amount;
a pulse wave detecting step of detecting a pulse wave of a blood flow of the person from the image signal of the region of the face detected in the face detecting step;
a scoring step of calculating a score of the expression of the person based on the expression feature amount;
a coaching step of generating an expression guide that induces a change in the expression of the person so as to improve the score; and
a displaying step of displaying the expression guide,
wherein the displaying step further includes a step of displaying biological information indicating a state of an autonomic nerve of the person calculated based on the pulse wave after displaying the expression guide, and the score calculated based on the expression feature amount after displaying the expression guide.
